(19) United States
(12) Patent Application Publication
(10) Pub. No.: US 2014/0249445 A1
(43) Pub. Date: Sep. 4, 2014

(54) Methods and Devices for Improving Cognitive Function

(71) Applicant: Wake Forest University Health Sciences, Winston-Salem, NC (US)

(72) Inventors: Sam Deadwyler, Winston-Salem, NC (US); Robert E. Hampson, Kernersville, NC (US); Mark R. Witcher, Winston-Salem, NC (US); Daniel E. Couture, Winston-Salem, NC (US); Gautam Popli, Winston-Salem, NC (US); Stephen Tatter, Winston-Salem, NC (US); Thomas L. Ellis, Winston-Salem, NC (US)

(73) Assignee: Wake Forest University Health Sciences, Winston-Salem, NC (US)

(21) Appl. No.: 14/196,707

(22) Filed: Mar. 4, 2014

Related U.S. Application Data
(60) Provisional application No. 61/772,365, filed on Mar. 4, 2013.

(51) Int. Cl. A61N 1/36; A61B 5/0476

(52) U.S. Cl. CPC A61N 1/36025; A61B 5/0476; A61N 1/36082
USPC 600/544; 607/45

(57) ABSTRACT

The present invention provides methods and devices for improving cognitive function. In some embodiments, cognitive function is improved by detecting neuronal activity in the CA3 region of a patient's hippocampus and stimulating the CA3 region of the patient's hippocampus responsive to the neuronal activity detected.

### RELATED APPLICATIONS

The present application claims the benefit of priority under 35 U.S.C. §119(e) to U.S. Provisional Patent Application No. 61/772,365, filed Mar. 4, 2013, the disclosure of which is incorporated herein by reference in its entirety.

### FIELD OF THE INVENTION

The present invention concerns apparatuses and associated methods useful for improving cognitive function.

### BACKGROUND

Epilepsy is a common chronic neurologic disorder which causes recurrent seizures and affects 0.5 to 1 percent of the population (Hauser et al., EPILEPSIA 34:453 (1993)). Seizures occur when there is an imbalance in the electrical activity of the brain. The abnormality may be in a small area of the brain or may involve the entire brain. Epilepsy can be congenital or can also be caused by head injury, birth trauma, infections, brain tumor, hemorrhage, or stroke. Diagnosis of epilepsy requires multi-modality testing, often in multiple phases. Initially, clinical exam and patient history are of primary importance for diagnosis. Other tests may include magnetic resonance imaging (MRI) to examine for structural abnormalities, single photon emission computed tomography (SPECT or positron emission tomography (PET) to examine for functional abnormality, electroencephalography (EEG) to record electrical impulses of the brain and their foci, magnetoencephalography (MEG) to co-localize electrical foci with structural abnormalities, and blood tests to exclude other diseases.

In spite of optimal medical management, more than one-third of all epilepsy patients have incompletely controlled seizures or debilitating medication side effects (Kwan and Brodie, N. ENGL. J. MED. 342:314 (2000); Sander, EPILEPSIA 34:1007 (1993); Sillanpaa and Schmidt, EPILEPSY BEHAV. 8:713 (2006)). Medically refractory epilepsy is associated with excess injury and mortality, psychosocial dysfunction, and significant cognitive impairment. Resective or disconnective surgery is associated with long term seizure freedom in 60-80% of patients (Engel, Jr. et al., EPILEPSIA 44:741 (2003); Engel, Jr. et al., NEUROL. 60:538 (2003); Lee et al., ANN. NEUROL. 58:525 (2005)). Surgery for patients whose epilepsy has proven refractory to anti-epileptic drugs (AEDs) provides a high likelihood of reduction in seizure frequency, is generally safe, and is recommended for selected patients with refractory partial seizures.

In order to appropriately diagnose epilepsy for possible surgical intervention, it is generally necessary to conduct an inpatient workup, referred to as Phase I monitoring. The primary goal of this analysis is to confirm the diagnosis of epilepsy and requires simultaneous video and electroencephalographic (EEG) monitoring, 24 hours a day in an epilepsy monitoring unit (EMU), which enables correlation of complete clinical behavior, with seizure information from the EEG. Through Phase I monitoring, the part of the brain responsible for seizure activity can often be localized. However, if data collected during the Phase I admission does not provide enough information to localize the seizure focus, then invasive, inpatient, Phase II monitoring may be needed. This standard approach involves surgery to place cortical electrodes and depth electrodes directly into the brain. Cortical electrodes consist of parallel rows of electrode contacts placed directly on the cortical surface of the brain. Depth electrodes are inserted into the brain to reach deep recording sites, often in the hippocampus and amygdala. These electrodes are placed to provide more accurate information as to the location of epileptic focus and to correlate with continuous video monitoring. After recovery from surgery, implanted electrodes are connected to monitoring equipment which can detect seizure activity and localize seizure foci. In order to provoke seizures, anti-epileptic medications are often weaned or discontinued.

### The Hippocampus in Cognition and Memory Formation

Humans are unique in exhibiting a vast array of higher thought processes, representational skills, and computational capabilities. The cortical brain systems underlying cognitive function are intimately involved in neurological disorders and neurodegenerative diseases, and display a high susceptibility to neural damage in pathologic conditions such as epilepsy, traumatic brain injury, Alzheimer's disease, mild cognitive impairment. Given the uniqueness of cognitive processes in brain function, in contrast to sensory and motor function, the hierarchical intricacies of human brain cytoarchitecture and synaptic organization, the singular role of cognition defines what it is to be “human”. Critical to this role is the fundamental need to better understand the neural substrates underlying human cognition and memory formation. Namely, we must better understand the in vive function of the human hippocampus.

### Hippocampal Subfield Anatomy

In brief, the anatomical hippocampus is subdivided into subfields known as the CA1-CA4 regions, each populated by a stereotypical cytoarchitecture with predictable synaptic interconnections between subfields (DUVERNOY, THE HUMAN HIPPOCAMPUS: FUNCTIONAL ANATOMY, VASCULARIZATION AND SERIAL SECTIONS WITH MRI (2005)). Importantly, the subfield of CA3 gives putative monosynaptic input to the CA1 subfield via a collection of pyramidal neuronal axons known as the Schaffer collateral system (Schaffer, ARCH. MIKROSKOP. ANATOMIE 39:611 (1892)); the CA1 subfield subsequently serves in the main output pathway of the hippocampal formation. In the past several years, much attention has focused on how the firing pattern of CA3 neuronal population influences the neuronal discharge of the CA1 subfield (Guzowski et al., NEURON 44:581 (2004)) leading recently to the development of verified animal models which reliably predict multineuronal ensemble firing patterns of CA1 based on the recorded discharge patterns of CA3 (Berger et al., J. NEURONAL ENG. 8:046017 (2011); Hampson et al., IEEE TRANSACTIONS 20:184 (2012)).

Recording of these hippocampal ensembles for use in functional predictions of seizures can now be readily adapted to human application using FDA-approved materials within a well-defined clinical application.

Hippocampal Recording and Development of Neuronal Ensemble Models from Animal Studies

As described above, the hippocampus has a stereotypical cytoarchitecture which includes a putative monosynaptic connection between subfields (DUVERNOY, THE HUMAN HIPPOCAMPUS: FUNCTIONAL ANATOMY, VASCULARIZATION AND SERIAL SECTIONS WITH MRI (2005)). The neuronal output of CA3 has recently been shown in mathematical models of animal hippocampus to influence the neuronal activity pattern of CA1 neurons, forming a neuronal ensemble. In the rodent hippocampus, one such operational nonlinear systems model characterized the neuronal activity of CA1 utilizing individualized or generic inputs derived from CA3 neurons, recorded while the animals underwent cognitive behavioral testing (Berger et al., J. NEURONAL ENG. 8:046017 (2011); Hampson et al., IEEE TRANSACTIONS 20:184 (2012); Zanos et al., IEEE TRANSACTIONS 16:336 (2008)). Development of this model has been shown to allow reliable recording of CA3 neuronal discharges with subsequent accurate prediction of the activity of CA1 postsynaptic cells via the Schaffer collateral system (Berger et al., J. NEURONAL ENG. 8:046017 (2011)).

### SUMMARY OF THE CLAIMED INVENTION

A first aspect of the invention is a method of improving cognitive function. In some embodiments, the method comprises, consists essentially of or consists of delivering one or more stimuli to a patient's brain to improve cognitive function. In some embodiments, cognitive function is improved by stimulating the CA3 region of the patient's hippocampus, the Schaffer collateral region of the patient's hippocampus, the CA1 region of the patient's hippocampus, the CA2 region of the patient's hippocampus, the patient's perforant pathway, the patient's subiculum, the patient's entorhinal cortex and/or the patient's temporal cortex (with an electrical stimulus delivered by one or more electrodes, for example). In some embodiments, the method further comprises detecting and/or analyzing neuronal activity in the patient's brain. In some such embodiments, neuronal activity in the CA3 region of the patient's hippocampus, the Schaffer collateral region of the patient's hippocampus, the CA1 region of the patient's hippocampus, the CA2 region of the patient's hippocampus, the patient's perforant pathway, the patient's subiculum, the patient's entorhinal cortex and/or the patient's temporal cortex is detected and/or analyzed prior to, during and/or following said delivering step.

A second aspect of the present invention is a device useful for improving cognitive function. In some embodiments, the device comprises, consists essentially of or consists of a controller operatively connected to one or more electrodes (e.g., one or more single-unit neuron detectors and/or one or more multi-unit neuron detectors), wherein at least one of the one or more electrodes is configured to detect neuronal activity in the CA3 region of a patient's hippocampus and wherein the controller is configured to stimulate one or more hippocampal neuronal ensembles based upon neuronal activity detected in the CA3 region of the patient's hippocampus and/or a connected region of the brain. In some embodiments, the device comprises, consists essentially of or consists of a controller operatively connected to a plurality of electrodes (e.g., a plurality of single-unit neuron detectors and/or a plurality of multi-unit neuron detectors), wherein one or more of the electrodes is configured to detect neuronal activity in the CA3 region of a patient's hippocampus and one or more of the plurality of electrodes is configured to detect neuronal activity in an alternate region of the patient's brain (e.g., the Schaffer collateral and/or CA1 regions of the patient's hippocampus, the subiculum and/or the entorhinal cortex) and wherein the controller is configured to stimulate one or more hippocampal neuronal ensembles based upon neuronal activity detected in the CA3 region of the patient's hippocampus and the alternate region of the patient's brain.

The foregoing and other objects and aspects of the present invention are explained in greater detail in the drawings herein and the specification set forth below.

### BRIEF DESCRIPTION OF THE DRAWINGS

FIG. 1 is a block diagram of a device according to some embodiments of the present invention.

FIG. 2 is a block diagram of a controller according to some embodiments of the present invention.

### DETAILED DESCRIPTION

The present invention is explained in greater detail below. This description is not intended to be a detailed catalog of all the different ways in which the invention may be implemented or of all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant invention, will be apparent to those skilled in the art in light of the instant disclosure. Hence, the following specification is intended to illustrate some particular embodiments of the invention, and not to exhaustively specify all permutations, combinations and variations thereof.

All patents, patent publications and non-patent publications referenced herein are incorporated by reference in their entireties.

It is to be understood that when an element or layer is referred to as being “on”, “attached to”, “connected to”, “coupled to”, “coupled with” or “contacting” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. It will also be appreciated by those of skill in the art that references to a structure or feature that is disposed “adjacent” another structure or feature may have portions that overlap or underlie the adjacent structure or feature.

The present invention is described below with reference to block diagrams and/or flowchart illustrations of methods, systems and/or computer program products according to embodiments of the invention.

It is to be understood that various blocks of the block diagrams and/or flowchart illustrations, and combinations of blocks in the block diagrams and/or flowchart illustrations, can be implemented by computer program instructions. These computer program instructions may be provided to a processor of a general purpose computer, special purpose computer and/or other programmable data processing apparatus to produce a machine, such that the instructions, which execute via the processor of the computer or other programmable data processing apparatus, create means for implementing the functions/acts specified in the block diagrams and/or flowchart illustrations. The computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagram and/or flowchart illustrations. The computer program instructions may also be loaded onto a computer or other programmable data processing apparatus to cause a series of operational steps to be performed on the computer or other programmable data processing apparatus to produce a computer-implemented process such that the instructions which execute on the computer or other programmable data processing apparatus provide steps for implementing the functions/acts specified in the block diagrams and/or flowchart illustrations.

Accordingly, the present invention may be embodied in hardware and/or software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system. In the context of this document, a computer usable or computer-readable non-transient storage medium may be any medium that can contain and/or store the program for use by or in connection with the instruction execution system, apparatus or device. For example, the computer-usable or computer-readable medium may be an electronic, optical, electromagnetic, infrared or semiconductor system, apparatus or device.

The terminology used herein is for the purpose of describing particular embodiments only and is not intended to be limiting of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

As used herein, the singular forms “a”, “an” and “the” are intended to include the plural forms as well, unless the context clearly indicates otherwise.

As used herein, the term “adjuvant treatment” refers to a treatment modifies the effect(s) of one or more other treatments (e.g., one or more pharmaceutical agents). For example, the delivery of one or more stimuli to the CA3 region of a patient's hippocampus may enhance the effectiveness of a pharmaceutical agent (by restoring the therapeutic efficacy of a drug to which the patient had previously become habituated, for example). In some embodiments, delivery of one or more stimuli to the patient's brain may reduce or eliminate the need for one or more treatments (e.g., one or more pharmaceutical agents).

As used herein, the term “and/or” includes any and all combinations of one or more of the associated listed items.

As used herein, the term “cognitive activity” refers to an electrical discharge in the brain resulting from conscious intellectual activity (e.g., thinking, reasoning and/or remembering).

As used herein, the terms “comprise,” “comprises,” “comprising,” “include”, “includes” and “including” specify the presence of stated features, integers, steps, operations, elements, and/or components, but do not preclude the presence or addition of one or more other features, integers, steps, operations, elements, components, and/or groups thereof.

As used herein, the term “neuronal activity” refers to electrical impulses generated by one or more neurons. Thus, “detect neuronal activity” (and grammatical variants thereof) refers to the detection of electrical impulses generated by one or more neurons (using one or more implantable electrodes, for example).

As used herein, the term “patient” refers to both human subjects and animal subjects, including, but not limited to, mice, rats, rabbits, cats, dogs, pigs, horses, monkeys, apes, etc. The patient may be male or female. That patient may be of any suitable age, including infant, juvenile, adolescent, adult and geriatric ages. In some embodiments, the methods, devices and systems of the present invention may be used to induce physiological and/or psychological responses in a patient for medically diagnostic and/or therapeutic purposes. For example, the methods, devices and systems of the present invention may be used to diagnose and/or treat mammalian subjects, such as mice, rats, pigs and monkeys, for medical research or veterinary purposes.

As used herein, the terms “treatment,” “treat,” and “treating” refer to reversing, alleviating, reducing the severity of, delaying the onset of, inhibiting the progress of or preventing a disease or disorder as described herein, or at least one symptom of a disease or disorder as described herein (e.g., inhibiting and/or preventing one or more cognitive impairments). In some embodiments, treatment may be administered after one or more symptoms have developed (e.g., following the onset of memory loss). In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved—for example, to prevent or delay their recurrence. Treatment may be as an adjuvant treatment as further described herein.

Methods and devices of the present invention may be used to improve cognitive function.

Methods of the present invention comprise, consist essentially of or consist of delivering a stimulus to one or more regions of a patient's brain, thereby improving cognitive function. In some embodiments, the method further comprises detecting and/or analyzing neuronal activity in the patient. Neuronal activity may be detected and/or analyzed prior to, during and/or following delivery of the stimulus to said one or more regions of the patient's brain.

Any suitable method/device may be used to detect and/or analyze neuronal activity, including, but not limited to, the methods and devices described herein.

In some embodiments, the method comprises detecting neuronal activity in one or more regions of a patient's brain (e.g., one or more regions of a patient's hippocampus) and analyzing the detected neuronal activity.

In some embodiments, the method comprises detecting neuronal activity in the CA3 region of a patient's hippocampus and analyzing the detected neuronal activity.

In some embodiments, the method comprises detecting neuronal activity in the CA3 region of a patient's hippocampus and at least one alternate region of the patient's brain and analyzing the detected neuronal activity. The at least one alternate region may comprise one or more of the following: the Schaffer collateral region of the hippocampus, the CA1 region of the hippocampus, the CA2 region of the hippocampus, the amygdala, the perforant pathway, the subiculum, the entorhinal cortex, a neocortical region (e.g., a neocortical region of the temporal, frontal, parietal or occipital lobe), the cerebellum and the cingulate cortex. Thus, in some embodiments, the method comprises, consists essentially of or consists of detecting neuronal activity in the CA3 and CA1 regions of a patient's hippocampus and analyzing the detected neuronal activity. Similarly, in some embodiments, the method comprises, consists essentially of or consists of detecting neuronal activity in the CA3 and Schaffer collateral regions of a patient's hippocampus and analyzing the detected neuronal activity.

In some embodiments, the method comprises detecting neuronal activity in the Schaffer collateral region of a patient's hippocampus and analyzing the detected neuronal activity.

In some embodiments, the method comprises detecting neuronal activity in the Schaffer collateral region of a patient's hippocampus and at least one alternate region of the patient's brain and analyzing the detected neuronal activity. The at least one alternate region may comprise one or more of the following: the CA3 region of the hippocampus, the CAL region of the hippocampus, the CA2 region of the hippocampus, the amygdala, the perforant pathway, the subiculum, the entorhinal cortex and, a neocortical region (e.g., a neocortical region of the temporal, frontal, parietal or occipital lobe), the cerebellum and the cingulate cortex. Thus, in some embodiments, the method comprises, consists essentially of or consists of detecting neuronal activity in the Schaffer collateral and CA1 regions of a patient's hippocampus and analyzing the detected neuronal activity. Similarly, in some embodiments, the method comprises, consists essentially of or consists of detecting neuronal activity in the CA3 and Schaffer collateral regions of a patient's hippocampus and analyzing the detected neuronal activity.

In some embodiments, the method comprises detecting neuronal activity in the CA region of a patient's hippocampus and analyzing the detected neuronal activity.

In some embodiments, the method comprises detecting neuronal activity in the CA1 region of a patient's hippocampus and at least one alternate region of the patient's brain and analyzing the detected neuronal activity. The at least one alternate region may comprise one or more of the following: the CA3 region of the hippocampus, the Schaffer collateral region of the hippocampus, the CA2 region of the hippocampus, the amygdala, the perforant pathway, the subiculum, the entorhinal cortex, a neocortical region (e.g., a neocortical region of the temporal, frontal, parietal or occipital lobe), the cerebellum and the cingulate cortex. Thus, in some embodiments, the method comprises, consists essentially of or consists of detecting neuronal activity in the CA3 and CA1 regions of a patient's hippocampus and analyzing the detected neuronal activity. Similarly, in some embodiments, the method comprises, consists essentially of or consists of detecting neuronal activity in the CA1 and Schaffer collateral regions of a patient's hippocampus and analyzing the detected neuronal activity.

In some embodiments, the method comprises detecting neuronal activity in the CA2 region of a patient's hippocampus and analyzing the detected neuronal activity.

In some embodiments, the method comprises detecting neuronal activity in the CA2 region of a patient's hippocampus and at least one alternate region of the patient's brain and analyzing the detected neuronal. The at least one alternate region may comprise one or more of the following: the CA3 region of the hippocampus, the Schaffer collateral region of the hippocampus, the CA1 region of the hippocampus, the amygdala, the perforant pathway, the subiculum, the entorhinal cortex, a neocortical region (e.g., a neocortical region of the temporal, frontal, parietal or occipital lobe), the cerebellum and the cingulate cortex. Thus, in some embodiments, the method comprises, consists essentially of or consists of detecting neuronal activity in the CA3 and CA2 regions of a patient's hippocampus and analyzing the detected neuronal activity. Similarly, in some embodiments, the method comprises, consists essentially of or consists of detecting neuronal activity in the CA2 and Schaffer collateral regions of a patient's hippocampus and analyzing the detected neuronal activity. Likewise, in some embodiments, the method comprises, consists essentially of or consists of detecting neuronal activity in the CA2 region of a patient's hippocampus and at least one associated cortical region and analyzing the detected neuronal activity.

In some embodiments, the method comprises detecting neuronal activity in a patient's perforant pathway and analyzing the detected neuronal activity.

In some embodiments, the method comprises detecting neuronal activity in a patient's perforant pathway and at least one alternate region of the patient's brain and analyzing the detected neuronal activity. The at least one alternate region may comprise one or more of the following: the CA3 region of the hippocampus the Schaffer collateral region of the hippocampus, the CA1 region of the hippocampus, the CA2 region of the hippocampus, the amygdala, the subiculum, the entorhinal cortex, a neocortical region (e.g., a neocortical region of the temporal, frontal, parietal or occipital lobe), the cerebellum and the cingulate cortex. Thus, in some embodiments, the method comprises, consists essentially of or consists of detecting neuronal activity in a patient's perforant pathway and at least one region of the patient's hippocampus and analyzing the detected neuronal activity. Similarly, in some embodiments, the method comprises, consists essentially of or consists of detecting neuronal activity a patient's perforant pathway and the patient's subiculum and analyzing the detected neuronal activity. Likewise, in some embodiments, the method comprises, consists essentially of or consists of detecting neuronal activity in a patient's perforant pathway and the patient's entorhinal cortex and analyzing the detected neuronal activity.

In some embodiments, the method comprises detecting neuronal activity in a patient's subiculum and analyzing the detected neuronal activity.

In some embodiments, the method comprises detecting neuronal activity in a patient's subiculum and at least one alternate region of the patient's brain and analyzing the detected neuronal activity. The at least one alternate region may comprise one or more of the following: the CA3 region of the hippocampus the Schaffer collateral region of the hippocampus, the CA1 region of the hippocampus, the CA2 region of the hippocampus, the amygdala, the perforant pathway, the entorhinal cortex, a neocortical region (e.g., a neocortical region of the temporal, frontal, parietal or occipital lobe), the cerebellum and the cingulate cortex. Thus, in some embodiments, the method comprises, consists essentially of or consists of detecting neuronal activity in a patient's subiculum and at least one region of the patient's hippocampus and analyzing the detected neuronal activity. Similarly, in some embodiments, the method comprises, consists essentially of or consists of detecting neuronal activity a patient's subiculum and the patient's perforant pathway and analyzing the detected neuronal activity. Likewise, in some embodiments, the method comprises, consists essentially of or consists of detecting neuronal activity in a patient's subiculum and the patient's entorhinal cortex and analyzing the detected neuronal activity.

In some embodiments, the method comprises detecting neuronal activity in a patient's entorhinal cortex and analyzing the detected neuronal activity.

In some embodiments, the method comprises detecting neuronal activity in a patient's entorhinal cortex and at least one alternate region of the patient's brain and analyzing the detected neuronal activity. The at least one alternate region may comprise one or more of the following: the CA3 region of the hippocampus the Schaffer collateral region of the hippocampus, the CA1 region of the hippocampus, the CA2 region of the hippocampus, the amygdala, the perforant pathway, the subiculum, a neocortical region (e.g., a neocortical region of the temporal, frontal, parietal or occipital lobe), the cerebellum and the cingulate cortex. Thus, in some embodiments, the method comprises, consists essentially of or consists of detecting neuronal activity in a patient's entorhinal cortex and at least one region of the patient's hippocampus and analyzing the detected neuronal activity. Similarly, in some embodiments, the method comprises, consists essentially of or consists of detecting neuronal activity a patient's entorhinal cortex and the patient's perforant pathway and analyzing the detected neuronal activity. Likewise, in some embodiments, the method comprises, consists essentially of or consists of detecting neuronal activity in a patient's entorhinal cortex and the patient's subiculum and analyzing the detected neuronal activity.

Any suitable means may be used to detect neuronal activity, including, but not limited to, subdural electrodes and stereotactically-placed depth electrodes. In some embodiments, neuronal activity is detected using one or more single-unit neuron detectors (e.g., one or more microelectrodes) and/or one or more multi-unit neuron detectors (e.g., one or more macroelectrodes and/or one or more microelectrode arrays). In some embodiments, the method comprises, consists essentially of or consists of detecting neuronal activity in a patient's hippocampus (e.g., in the CA3, Schaffer collateral and/or CA1 regions of a patient's hippocampus) using one or more microelectrodes and/or one or more microelectrode arrays. Methods of the present invention may utilize electrodes with both microrecording capabilities and current recording capabilities, thereby allowing for the capture of single- and multi-unit neuronal ensemble patterns and the development of unique algorithmic models of the human brain and unique cognitive function models that are suitable for use in the improvement of cognitive function. For example, microelectrodes may be positioned in the CA3, Schaffer collateral and/or CA1 regions of the hippocampus to detect single-unit neuronal ensemble discharges, whilst macroelectrodes are used to detect neuronal discharges in the amygdala, hippocampus, perforant pathway, subiculum, entorhinal cortex and/or one or more neocortical regions, thereby allowing for the development of unique models for improving cognitive function.

The detected neuronal activity may be analyzed using any suitable methods/devices, including, but not limited to, the methods/device describe herein and the methods/devices described in U.S. Pat. No. 7,460,904, Berger et al., J. NEURONAL ENG. 8:046017 (2011); Brown et al., NEURAL COMPUTATION 14:346 (2002); Granger, ECONOMETRICA 37:424 (1969); Hampson et al., IEEE TRANSACTIONS 20:184 (2012); Hampson et al., J. NEUROSCI METHODS 182:195 (2009); Song et al., NEURAL NETWORKS 22:1340 (2009); Zanos et al., IEEE TRANSACTIONS 16:336 (2008), the disclosure of each of which is incorporated herein by reference in its entirety.

Any suitable aspect of the neuronal activity may be analyzed, including, but not limited to, the waveform, frequency and rhythmicity of the neuronal activity. In some embodiments, the presence and/or absence of rhythmic unit bursting (e.g., rhythmic unit bursting that coincides with increases/decreases in local field potentials) in one or more regions of the patient's brain (e.g., one or more regions of the patient's hippocampus) may be analyzed. In some embodiments, the recruitment of neurons into one or more a rhythmic firing patterns may be analyzed. For example, in some embodiments, the analysis focuses on detecting the occurrence of rhythmical single- and/or multi-unit neuronal depolarization in one or more regions of the patient's brain (e.g., the CA3, Schaffer collateral and/or CA1 regions of the patient's hippocampus) that coincide spatiotemporally with local field potential increases and/or synchronize in discharge frequency.

In some embodiments, analyzing the detected neuronal activity comprises comparing the detected neuronal activity with neuronal activity detected prior to, during and/or following performance of a cognitive task. For example, in an embodiment comprising the detection of neuronal activity in the CA3 region of a patient's hippocampus, analyzing the detected neuronal activity may comprise comparing the neuronal activity detected in the CA3 region of the patient's hippocampus with neuronal activity that was detected in the CA3 region of the patient's hippocampus prior to, during and/or following performance of a cognitive task and/or with neuronal activity that was detected in the CA3 region(s) of one or more other patients prior to, during and/or following performance of a cognitive task. Likewise, in an embodiment comprising the detection of neuronal activity in the CA3 and CA1 regions of a patient's hippocampus, analyzing the detected neuronal activity may comprise comparing the neuronal activity detected in the CA3 and CA1 regions of the patient's hippocampus with neuronal activity that was detected in the CA3 and CA1 regions of the patient's hippocampus prior to, during and/or following performance of a cognitive task and/or with neuronal activity that was detected in the CA3 and CA1 regions of one or more other patients prior to, during and/or following performance of a cognitive task.

In some embodiments, analyzing the detected neuronal activity comprises comparing the detected neuronal activity with a neuronal activity signature associated with one or more types of cognitive activity. For example, in an embodiment comprising the detection of neuronal activity in the CA3 region of a patient's hippocampus, analyzing the detected neuronal activity may comprise comparing the neuronal activity detected in the CA3 region of the patient's hippocampus with a neuronal activity signature derived by aggregating the neuronal activity detected in the CA3 regions of a plurality of patient's prior to and/or during performance of a cognitive task. Likewise, in an embodiment comprising the detection of neuronal activity in the CA3 and CA1 regions of a patient's hippocampus, analyzing the detected neuronal activity may comprise comparing the neuronal activity detected in the CA3 and CA1 regions of the patient's hippocampus with a neuronal activity signature derived by aggregating the neuronal activity detected in the CA3 and CA1 regions of a plurality of patient's prior to and/or during performance of a cognitive task.

In some embodiments, analyzing the detected neuronal activity comprises predicting future neuronal activity based upon the detected neuronal activity (e.g., predicting that the detected neuronal activity will cause and/or will be followed by specified electrical discharges). For example, in an embodiment comprising the detection of neuronal activity in the CA3 region of a patient's hippocampus, analyzing the detected neuronal activity may comprise predicting future neuronal activity in the CA3 region of the patient's hippocampus, the Schaffer collateral region of the patient's hippocampus, the CA1 region of the patient's hippocampus, in the patient's perforant pathway, in the patient's subiculum and/or in the patient's entorhinal cortex based upon the neuronal activity detected in the CA3 region of the patient's hippocampus. Likewise, in an embodiment comprising the detection of neuronal activity in the CA3 and CA1 regions of a patient's hippocampus, analyzing the detected neuronal activity may comprise predicting future neuronal activity in the CA3 region of the patient's hippocampus, the Schaffer collateral region of the patient's hippocampus, the CA1 region of the patient's hippocampus, in the patient's perforant pathway, in the patient's subiculum and/or in the patient's entorhinal cortex based upon the neuronal activity detected in the CA3 and CA1 regions of the patient's hippocampus.

In some embodiments, predicting future neuronal activity based upon the detected neuronal activity comprises the use of a predictive algorithm. Any suitable algorithm may be used, including, but not limited to, nonlinear algorithms and multi-input multi-output algorithms. See, e.g., Berger et al., J. NEURONAL ENG. 8:046017 (2011); Hampson et al., IEEE TRANSACTIONS 20:184 (2012); Song et al., NEURAL NETWORKS 22:1340 (2009); Zanos et al., IEEE TRANSACTIONS 16:336 (2008). For example, in an embodiment comprising the detection of neuronal activity in the CA3 region of a patient's hippocampus, predicting future neuronal activity based upon the detected neuronal activity may comprise the use of a predictive algorithm that that predicts future neuronal activity in the CA3 region of the patient's hippocampus, the Schaffer collateral region of the patient's hippocampus, the CA1 region of the patient's hippocampus, in the patient's perforant pathway, in the patient's subiculum and/or in the patient's entorhinal cortex based upon the neuronal activity detected in the CA3 region of the patient's hippocampus. Likewise, in an embodiment comprising the detection of neuronal activity in the CA3 and CA1 regions of a patient's hippocampus, predicting future neuronal activity based upon the detected neuronal activity may comprise the use of a predictive algorithm that that predicts future neuronal activity in the CA3 region of the patient's hippocampus, the Schaffer collateral region of the patient's hippocampus, the CA1 region of the patient's hippocampus, in the patient's perforant pathway, in the patient's subiculum and/or in the patient's entorhinal cortex based upon the neuronal activity detected in the CA3 and CA1 regions of the patient's hippocampus.

Any suitable stimulus may be delivered, including, but not limited to, electrical impulses.

A stimulus may be delivered to any suitable region of the patient's brain, including, but not limited to, the CA3 region of the hippocampus, the Schaffer collateral region of the hippocampus, the CA1 region of the hippocampus, the CA2 region of the hippocampus, the amygdala, the perforant pathway, the subiculum, the entorhinal cortex, the cerebellum, the cingulate cortex and neocortical regions, including, but not limited to, neocortical regions of the temporal, frontal, parietal and occipital lobes and/or any connections between two or more of the aforementioned regions. Thus, in some embodiments, the method comprises detecting and/or analyzing neuronal activity in a patient and delivering one or more stimuli to the CA3 region of a patient's hippocampus, one or more stimuli to the Schaffer collateral region of the patient's hippocampus, one or more stimuli to the CA1 region of the patient's hippocampus, one or more stimuli to the CA2 region of the patient's hippocampus, one or more stimuli to the patient's perforant pathway, one or more stimuli to the patient's subiculum and/or one or more stimuli to the patient's entorhinal cortex.

Any suitable device may be used to carry out the aforementioned methods, including, but not limited to, the devices described hereinafter.

As noted above, the present invention also provides devices useful for improving cognitive function.

In some embodiments, the device comprises, consists essentially of or consists of a controller that is operatively connected to one or more electrodes.

In some embodiments, the device comprises, consists essentially of or consists of a controller that is operatively connected to one or more electrodes configured to detect neuronal activity in the CA3 region of a patient's hippocampus, one or more electrodes configured to detect neuronal activity in the Schaffer collateral region of a patient's hippocampus, one or more electrodes configured to detect neuronal activity in the CA1 region of a patient's hippocampus, one or more electrodes configured to detect neuronal activity in the CA2 region of a patient's hippocampus, one or more electrodes configured to detect neuronal activity in a patient's perforant pathway, one or more electrodes configured to detect neuronal activity in a patient's subiculum, one or more electrodes configured to detect neuronal activity in a patient's entorhinal cortex and/or one or more electrodes configured to detect neuronal activity in a cortical area that is associated with one or more of the aforementioned regions/structures (e.g., a cortical area that receives input from one or more of the aforementioned regions/structures).

In some embodiments, the device comprises, consists essentially of or consists of a controller that is operatively connected to one or more electrodes configured to deliver one or more stimuli to the CA3 region of a patient's hippocampus, one or more electrodes configured to deliver one or more stimuli to the Schaffer collateral region of a patient's hippocampus, one or more electrodes configured to deliver one or more stimuli to the CA1 region of a patient's hippocampus, one or more electrodes configured to deliver one or more stimuli to the CA2 region of a patient's hippocampus, one or more electrodes configured to deliver one or more stimuli to a patient's perforant pathway, one or more electrodes configured to deliver one or more stimuli to a patient's subiculum, one or more electrodes configured to deliver one or more stimuli to a patient's entorhinal cortex and/or one or more electrodes configured to deliver one or more stimuli to a cortical area that is associated with one or more of the aforementioned regions/structures (e.g., a cortical area that receives input from one or more of the aforementioned regions/structures).

Devices of the present invention may comprise any suitable electrode(s), including, but not limited to, subdural electrodes and stereotactically-placed depth electrodes. In some embodiments, the device comprises one or more single-unit neuron detectors (e.g., one or more microelectrodes) and/or one or more multi-unit neuron detectors (e.g., one or more macroelectrodes and/or on or more microelectrode arrays). In some embodiments, the device comprises one or more electrodes with microrecording capability and current recording capability. Such electrodes may be used while collecting conventional clinical data for analysis across multiple spatioteniporal scales. Moreover, as noted above such electrodes may be used to capture single- and multi-unit neuronal ensemble patterns, which may themselves be used to develop unique algorithmic models of the epileptic human brain and unique cognitive function models.

The electrode(s) may be configured so as to be insertable/implantable in any suitable location in a patient's brain, including, but not limited to, the hippocampal formation (e.g., dentate gyrus, hippocampus, subiculum), the perforant pathway, the entorhinal cortex and cortical areas associated with one or more of the aforementioned regions/structures (e.g., a cortical area that receives input from one or more of the aforementioned regions/structures). In some embodiments, the device comprises one or more electrodes configured so as to be insertable/implantable in a patient's hippocampus (e.g., into the CA1, CA2, CA3 and/or Schaffer collateral region(s) of a patient's hippocampus). In some embodiments, the device comprises one or more electrodes configured so as to be insertable/implantable in the CA3 region of a patient's hippocampus, one or more electrodes configured so as to be insertable/implantable in the Schaffer collateral region of a patient's hippocampus and/or one or more electrodes configured so as to be insertable/implantable in the CA1 region of a patient's hippocampus.

The electrode(s) may be configured to detect neuronal activity in any suitable location in a patient's brain, including, but not limited to, the hippocampal formation (e.g., dentate gyrus, hippocampus, subiculum), the perforant pathway the entorhinal cortex and cortical areas associated with one or more of the aforementioned regions/structures (e.g., a cortical area that receives input from one or more of the aforementioned regions/structures). In some embodiments, the device comprises one or more electrodes configured to detect neuronal activity in a patient's hippocampus (e.g., in the CA1, CA2, CA3 and/or Schaffer collateral region(s) of a patient's hippocampus). In some embodiments, the device comprises one or more electrodes configured to detect neuronal activity in the CA3 region of a patient's hippocampus, one or more electrodes configured to detect neuronal activity in the Schaffer collateral region of a patient's hippocampus and/or one or more electrodes configured to detect neuronal activity in the CA1 region of a patient's hippocampus.

The electrode(s) may be configured to transmit any suitable data to the controller, including, but not limited to, electrical signals corresponding to the detected neuronal activity. In some embodiments, the electrode(s) is/are configured to capture electrical impulses generated by one or more neurons and to transmit those electrical impulses to the controller.

The electrode(s) may be configured to deliver any suitable stimulus, including, but not limited to, electrical stimuli (e.g., one or more electrical pulses). In some embodiments, the device comprises one or more electrodes configured to deliver electrical stimuli that are of sufficient timing, pulse width, charge, frequency and/or intensity to improve cognitive function.

The electrode(s) may be configured to deliver one or more stimuli to any suitable location in a patient's brain, including, but not limited to, the hippocampal formation (e.g., dentate gyrus, hippocampus, subiculum), the perforant pathway the entorhinal cortex and cortical areas associated with one or more of the aforementioned regions/structures (e.g., a cortical area that receives input from one or more of the aforementioned regions/structures). In some embodiments, the device comprises one or more electrodes configured to deliver a stimulus to a patient's hippocampus (e.g., to the CA1, CA2, CA3 and/or Schaffer collateral region(s) of a patient's hippocampus). In some embodiments, the device comprises one or more electrodes configured to deliver a stimulus to the patient's cerebellum, one or more electrodes configured to deliver a stimulus to the patient's perforant pathway, one or more electrodes configured to deliver a stimulus to the CA3 region of a patient's hippocampus, one or more electrodes configured to deliver a stimulus to the Schaffer collateral region of a patient's hippocampus, one or more electrodes configured to deliver a stimulus to the CA1 region of a patient's hippocampus, one or more electrodes configured to deliver a stimulus to the patient's subiculum and/or one or more electrodes configured to deliver a stimulus to the patient's entorhinal cortex.

Devices of the present invention may comprise any suitable controller, including, but not limited to, controllers configured so as to be implanted in and/or attached to a patient's body. For example, in some embodiments, the device may comprise a controller as described in U.S. Pat. No. 7,460,904, the disclosure of which is incorporated herein by reference in its entirety.

The controller may be operatively connected to the electrode(s) in any suitable manner. For example, the controller may be operatively connected to the electrode(s) via a detection lead and/or a stimulation lead. In those embodiments wherein the controller is operatively connected to a plurality of electrodes, the controller may be connected to each electrode separately (e.g., via a separate lead or set of leads). Individual leads and/or sets of leads may be bundled together to form one or more lead bundles. Leads, sets of leads and/or lead bundles may be operatively connected to the controller via a lead interface (e.g., an 18-pin connector).

The controller may be configured to receive data from any of the electrode(s) to which it is operatively connected. For example, the controller may be configured to receive data from one or more electrodes inserted/implanted in the perforant pathway, one or more electrodes inserted/implanted in the hippocampal formation (e.g., in the hippocampus and/or the subiculum), one or more electrodes inserted/implanted in the entorhinal cortex and/or one or more electrodes inserted/implanted in a cortical area associated with one or more of the aforementioned regions/structures (e.g., a cortical area that receives input from one or more of the aforementioned regions/structures). In some embodiments, the controller is configured to receive data from one or more electrodes inserted/implanted in the CA3 region of a patient's hippocampus, one or more electrodes inserted/implanted in the Schaffer collateral region of a patient's hippocampus and/or one or more electrodes inserted/implanted in the CA1 region of a patient's hippocampus.

The controller may be configured to receive any suitable data from the electrode(s) to which it is operatively connected, including, but not limited to, electrical signals corresponding to the neuronal activity detected by the electrode(s). In some embodiments, the controller is configured to receive electrical impulses captured by the electrode(s).

The controller may be configured to perform any suitable analysis responsive to receiving data from one or more electrodes, including, but not limited to, predicting whether/when a cognitive activity will occur, predicting the intensity of a cognitive activity, predicting the duration of a cognitive activity, detecting the onset of a cognitive activity, detecting an ongoing cognitive activity and/or detecting the cessation of a cognitive activity. In some embodiments, the controller is configured to analyze data associated with neuronal activity using a predictive algorithm. See, e.g., Berger et al., J. NEURONAL ENG. 8:046017 (2011); Granger, ECONOMETRICA 37:424 (1969); Hampson et al., IEEE TRANSACTIONS 20:184 (2012); Song et al., NEURAL NETWORKS 22:1340 (2009); Zanos et al., IEEE TRANSACTIONS16:336 (2008). In some embodiments, the controller is configured to analyze data associated with neuronal activity using an algorithm that predicts neuronal activity in the CA1 region of the hippocampus, the subiculum and/or the entorhinal cortex based upon neuronal activity detected in the CA3 and/or the Schaffer collateral regions of the hippocampus. In some embodiments, the controller is configured to analyze data associated with neuronal activity using an algorithm that predicts neuronal activity in cortical areas associated with the hippocampus formation based upon neuronal activity detected in the CA3, Schaffer collateral and/or CA1 regions of the hippocampus.

The controller may be configured to activate any of the electrode(s) to which it is operatively connected. For example, the controller may be configured to activate one or more electrodes inserted/implanted in the perforant pathway, one or more electrodes inserted/implanted in the hippocampal formation (e.g., in the hippocampus and/or the subiculum), one or more electrodes inserted/implanted the entorhinal cortex and/or one or more electrodes inserted/implanted in a cortical area associated with one or more of the aforementioned regions/structures (e.g., a cortical area that receives input from one or more of the aforementioned regions/structures). In some embodiments, the controller is configured to activate at least one electrode inserted/implanted in the patient's perforant pathway, at least one electrode inserted/implanted in the CA3 region of a patient's hippocampus, at least one electrode inserted/implanted in the Schaffer collateral region of a patient's hippocampus, at least one electrode inserted/implanted in the CA1 region of a patient's hippocampus, at least one electrode inserted/implanted in the patient's subiculum and/or at least one electrode inserted/implanted in the patient's entorhinal cortex.

The controller may be configured to activate one or more electrodes in response to any suitable trigger, including, but not limited to, detection of a cognitive activity. For example, the controller may be configured to activate one or more electrodes responsive to predicting that a cognitive activity is likely/imminent and/or predicting that activation of one or more electrodes may improve cognitive function. In some embodiments, the controller is configured to activate one or more electrodes responsive to detecting a cognitive activity in the patient's hippocampus (e.g., in the CA3, Schaffer collateral and/or CA1 regions of the hippocampus).

The controller may be configured to activate electrodes in any suitable manner. For example, the controller may be configured to activate an electrode such that the electrode delivers an electrical stimulus to a patient's brain (e.g., an electrical pulse). In some embodiments, the controller is configured to activate one or more electrodes to produce an electrical stimulus that enhances the propagation of neuronal activity from one lobe of the brain to another (e.g., from the temporal lobe to the frontal lobe). In some embodiments, the controller is configured to activate one or more electrodes to produce an electrical stimulus that enhances the propagation of neuronal activity within a particular lobe (e.g., from the hippocampus to the subiculum, from the subiculum to entorhinal cortex, etc.). In some embodiments, the controller is configured to activate one or more electrodes to produce an electrical stimulus that enhances the propagation of neuronal activity from one region of the hippocampus to another (e.g., from the CA3 region to the CA1 region).

The controller may be configured to receive and/or transmit data over any suitable wired or wireless communications channel, including, but not limited to, a LAN, the Internet, a public telephone switching network, Bluetooth, WLAN and the like.

In some embodiments, the controller comprises memory, a processor and a power supply.

As will be appreciated by one of skill in the art, the processor may be any commercially available or custom microprocessor. Memory can include, but is not limited to, the following types of devices: cache, ROM, PROM, EPROM, EEPROM, flash memory, SRAM and DRAM. The power supply may be an internal power supply (e.g., one or more rechargeable batteries that may be recharged without first being removed from the controller).

The controller's memory may comprise any suitable software and/or data, including, but not limited to, an operating system, applications, data and input/output (I/O) drivers.

As will be appreciated by one of skill in the art, the controller may use any suitable operating system, including, but not limited to, OS/2, AIX, OS/390 or System390 from International Business Machines Corp. (Armonk, N.Y.), Window CE, Windows NT, Windows95, Windows98, Windows2000, Windows 7 or Windows Vista from Microsoft Corp. (Redmond, Wash.), Mac OS from Apple, Inc. (Cupertino, Calif.), Unix, Linux or Android.

As will be appreciated by one of skill in the art, the controller may comprise any suitable application, including, but not limited to, one or more programs configured to implement one or more of the various features of the present invention.

For example, the controller may comprise a detection module configured to receive data associated with the detection of neuronal activity in one or more regions of a patient's brain; a prediction module configured to analyze data associated with the detection of neuronal activity in one or more regions of a patient's brain; an activation module configured to activate one or more electrodes; a network module configured to receive and/or transmit data and/or a safety module configured to deactivate the controller in the event of a system malfunction and/or failure. In some embodiments, two or more of the aforementioned modules are combined to form a single module configured to carry out the function(s) of each of the individual modules (e.g., the controller may comprise a detection-prediction module configured to receive and analyze data associated with the detection of neuronal activity in one or more regions of a patient's brain). In some embodiments, one of the aforementioned modules is split into two or more distinct modules. In some embodiments, one or more of the functions described below with respect to one of the modules described below is performed by one of the other modules described below.

In some embodiments, the controller comprises a detection module configured to receive and/or transmit data associated with the detection of neuronal activity in one or more regions of a patient's brain.

The detection module may be configured to receive and/or transmit data from/to any suitable device/module/database, including, but not limited to, one or more electrodes, other modules residing in the controller and databases residing in the controller. In some embodiments, the detection module is configured to receive data from one or more electrodes inserted/implanted in a patient's hippocampus (e.g., in the CA3, Schaffer collateral and/or CA1 regions of the patient's hippocampus), from one or more electrodes inserted/implanted in a patient's perforant pathway, from one or more electrodes inserted/implanted in a patient's subiculum, from one or more electrodes inserted/implanted in a patient's entorhinal cortex, from one or more electrodes inserted/implanted in a patient's cingulate cortex, from one or more electrodes inserted/implanted in a patient's cerebellum, from one or more electrodes inserted/implanted in a patient's neocortex (e.g., one or more electrodes inserted/implanted in a neocortical region of the patient's temporal, frontal, parietal and/or occipital lobes) and/or from a detection database residing in the controller. In some embodiments, the detection module is configured to transmit data to a detection database residing in the controller and/or to a prediction module residing in the controller.

The detection module may be configured to receive and/or transmit any suitable data, including, but not limited to, electrical signals captured by one or more electrodes inserted/implanted in a patient's brain. In some embodiments, the detection module is configured to receive and/or transmit electrical signals captured by one or more electrodes inserted/implanted in the one or more regions of a patient's hippocampus (e.g., the CA3, Schaffer collateral and/or CA1 regions of the patient's hippocampus).

In some embodiments, the controller comprises a prediction module configured to analyze data associated with the detection of neuronal activity in one or more regions of a patient's brain.

The prediction module may be configured to receive and/or transmit data from/to any suitable device/module/database, including, but not limited to, other modules residing in the controller and databases residing in the controller. In some embodiments, the prediction module is configured to receive data from a detection module residing in the controller and/or from a detection database residing in the controller. In some embodiments, the prediction module is configured to transmit data to a prediction database residing in the controller and/or to an activation module residing in the controller.

The prediction module may be configured to receive, transmit and/or analyze any suitable data, including, but not limited to, data associated with the detection of neuronal activity in one or more regions of a patient's brain and data associated with its own analysis. In some embodiments, the prediction module is configured to receive and analyze data associated with the detection of neuronal activity in one or more regions of a patient's hippocampus (e.g., the CA3, Schaffer collateral and/or CA1 regions of the patient's hippocampus), one or more regions of a patient's perforant pathway, one or more regions of a patient's subiculum, one or more regions of a patient's entorhinal, one or more regions of a patient's cerebellum, one or more regions of a patient's cingulate cortex and/or one or more regions of a patient's neocortex (e.g., one or more regions of the patient's temporal, frontal, parietal and/or occipital lobes).

The prediction module may be configured to analyze electrical signals in any suitable manner. In some embodiments, the prediction module is configured to analyze data associated with neuronal activity using a predictive algorithm. See, e.g., Berger et al., J. NEURONAL ENG. 8:046017 (2011); Granger, ECONOMETRICA 37:424 (1969); Hampson et al., IEEE TRANSACTIONS 20:184 (2012); Song et al., NEURAL NETWORKS 22:1340 (2009); Zanos et al., IEEE TRANSACTIONS 16:336 (2008). In some embodiments, the prediction module is configured to analyze data associated with neuronal activity using an algorithm that predicts neuronal activity in the CA1 region of the hippocampus, the perforant pathway, the subiculum, the entorhinal cortex and/or one or more neocortical regions (e.g., a neocortical region of the temporal, frontal, parietal or occipital lobe) based upon neuronal activity detected in the CA3 and/or the Schaffer collateral regions of the hippocampus, in the cingulate cortex, in the cerebellum, etc. In some embodiments, the prediction module is configured to analyze data associated with neuronal activity using an algorithm that predicts neuronal activity in cortical areas associated with the hippocampus formation based upon neuronal activity detected in the CA3, Schaffer collateral and/or CA1 regions of the hippocampus. In some embodiments, the prediction module is configured to predict whether/when a cognitive activity will occur, to predict the intensity of a cognitive activity, to predict the duration of a cognitive activity, to detect the onset of a cognitive activity, to detect an ongoing cognitive activity and/or to detect the cessation of a cognitive activity.

In some embodiments, the controller comprises an activation module configured to activate one or more electrodes (i.e., to control the magnitude, duration and other attributes of stimulation delivered by the electrode(s)).

The activation module may be configured to receive and/or transmit data from/to any suitable device/module/database, including, but not limited to, other modules residing in the controller and databases residing in the controller. In some embodiments, the activation module is configured to receive data from a prediction module residing in the controller and/or from an instruction database residing in the controller.

The activation module may be configured to receive and/or transmit any suitable data, including, but not limited to, instructions for activating one or more electrodes. In some embodiments, the activation module is configured to receive instructions for activating one or more electrodes inserted/implanted in a patient's hippocampus (e.g., in the CA3, Schaffer collateral and/or CA1 regions of the patient's hippocampus), one or more electrodes inserted/implanted in a patient's perforant pathway, one or more electrodes inserted/implanted in a patient's subiculum, one or more electrodes inserted/implanted in a patient's entorhinal cortex, one or more electrodes inserted/implanted in a patient's cingulate cortex, one or more electrodes inserted/implanted in a patient's cerebellum and/or one or more electrodes inserted/implanted in a patient's neocortex (e.g., one or more electrodes inserted/implanted in a neocortical regions of the patient's temporal, frontal, parietal and/or occipital cortex).

The activation module may be configured to activate the electrode(s) to deliver any suitable stimulus, including, but not limited to, one or more electrical impulses. In some embodiments, the activation module is configured to selectively and separately activate a plurality of electrodes (e.g., by activating only one of said plurality of electrodes, by sequentially activating the electrodes, by activating different electrodes using different stimulus parameters, combinations of some or all of the foregoing, etc.). In some embodiments, the activation module is configured to activate the electrode(s) responsive to a trigger. For example, the activation module may be configured to activate one or more electrodes if/when a cognitive activity is predicted and/or detected (by a prediction module residing in the controller, for example). In some embodiments, the activation module is configured to adjust one or more attributes of electrode activation (e.g., magnitude, duration, etc.) in response to feedback received from one or more electrodes and/or one or more sensors.

In some embodiments, the controller comprises a network module configured to receive, retrieve and/or transmit data.

The network module may be configured to receive, retrieve and/or transmit data from/to any suitable device/module/database, including, but not limited to, other modules residing in the controller and databases residing in the controller. In some embodiments, the network module is configured to receive data from a detection module residing in the controller, from a detection database residing in the controller, from a prediction module residing in the controller, from a prediction module residing in the controller and/or from an instruction database residing in the controller. In some embodiments, the network module is configured to transmit data to a detection database residing in the controller, to a prediction module residing in the controller, to a prediction module residing in the controller, to an instruction database residing in the controller and/or to an activation module residing in the controller.

The network module may be configured to receive, retrieve and/or transmit any suitable data, including, but not limited to, data associated with the detection of neuronal activity in one or more regions of a patient's brain, data associated with the analysis of data associated with the detection of neuronal activity in one or more regions of a patient's brain, data associated with one or more predicted/detected cognitive activities and instructions for activating one or more electrodes. In some embodiments, the network module is configured to receive and/or retrieve data associated with the detection of neuronal activity in one or more regions of a patient's brain from a detection module/database residing in the controller. In some embodiments, the network module is configured to receive and/or retrieve data associated with one or more predicted/detected cognitive activities from a prediction module/database residing in the controller. In some embodiments, the network module is configured to receive and/or retrieve instructions for activating one or more electrodes from a prediction module/database residing in the controller and/or from an instructions database. In some embodiments, the network module is configured to transmit data associated with the detection of neuronal activity in one or more regions of a patient's brain to a detection database residing in the controller and/or to prediction module residing in the controller. In some embodiments, the network module is configured to transmit data associated with one or more predicted/detected cognitive activities to a prediction database residing in the controller and/or to an activation module residing in the controller. In some embodiments, the network module is configured to transmit instructions for activating one or more electrodes to an instructions database residing in the controller and/or to an activation module residing in the controller.

In some embodiments, the controller comprises a safety module configured to deactivate the controller in the event of a system malfunction and/or failure.

The safety module may be configured to deactivate the controller for any suitable reason, including, but not limited to, a malfunctioning electrode, overheating and faulty activation of one or more electrodes by the controller. In some embodiments, the safety module is configured to deactivate the controller if/when the temperature of an electrode surpasses a specified safety threshold. In some embodiments, the safety module is configured to deactivate the controller if/when the temperature of the controller surpasses a specified safety threshold. In some embodiments, the safety module is configured to deactivate the controller if/when one or more of the activation signals sent from the controller to the associated electrode(s) indicates that the system may be operating outside of a predefined safety range. For example, the safety module may be configured to deactivate the controller if/when an activation signal sent from the controller to an associated electrode exceeds the level of activation that would normally be required to deliver the necessary stimulus in a properly functioning system.

As will be appreciated by one of skill in the art, the controller may comprise any suitable data, including, but not limited to, static and/or dynamic data used by the operating system, applications, I/O device drivers and other software components, data associated with the detection of neuronal activity in one or more regions of a patient's brain and instructions for activating one or more electrodes. For example, the controller may comprise a detection database comprising data associated with the detection of neuronal activity in one or more regions of a patient's brain, a prediction database comprising data associated with one or more predicted/detected cognitive activities and/or an instruction database comprising instructions for activating one or more electrodes. In some embodiments, two or more of the aforementioned databases are combined to form a single database comprising data from each of the individual databases (e.g., the controller may comprise a prediction-instruction database comprising data associated with one or more predicted/detected cognitive activities and instructions for activating one or more electrodes). In some embodiments, one of the aforementioned databases is split into two or more distinct databases. In some embodiments, one or more of the data types described below with respect to one of the databases described below is stored in one of the other databases described below. The controller may be configured to transmit, receive and store data in a manner that ensures compliance with any and all applicable laws and/or regulations (e.g., the Health Insurance Portability and Accountability Act of 1996 (P.L. 104-191; “HIPAA”)).

In some embodiments, the controller comprises a detection database configured to receive, store and/or transmit data associated with the detection of neuronal activity in one or more regions of a patient's brain (e.g, electrical signals captured by one or more electrodes inserted/implanted in the patient's brain).

The detection database may comprise any suitable type of memory including, but not limited to, cache, ROM, PROM, EPROM, EEPROM, flash memory, SRAM and DRAM.

The detection database may be configured to receive and/or transmit data from/to any suitable device/module/database, including, but not limited to, one or more electrodes and modules residing in the controller. In some embodiments, the detection database is configured to receive data from one or more electrodes implanted in a patient's hippocampus (e.g., in the CA3, Schaffer collateral and/or CA1 regions of the patient's hippocampus), from one or more electrodes implanted in a patient's perforant pathway, from one or more electrodes implanted in a patient's cingulate cortex, from one or more electrodes implanted in a patient's cerebellum, from one or more electrodes implanted in a patient's amygdalae, from one or more electrodes implanted in a patient's subiculum, from one or more electrodes implanted in a patient's entorhinal cortex, from one or more electrodes implanted in a patient's neocortex (e.g., in one or more neocortical regions of the patient's temporal, frontal, parietal and/or occipital lobes), from a detection module residing in the controller and/or from a prediction module residing in the controller. In some embodiments, the detection database is configured to transmit data to a detection module residing in the controller, to a prediction module residing in the controller and/or to an activation module residing in the controller.

In some embodiments, the controller comprises a prediction database configured to receive, store and/or transmit data associated with the detection of neuronal activity in one or more regions of a patient's brain, data associated with the analysis of data associated with the detection of neuronal activity in one or more regions of a patient's brain and/or data associated with one or more predicted/detected cognitive activities.

The prediction database may comprise any suitable type of memory including, but not limited to, cache, ROM, PROM, EPROM, EEPROM, flash memory, SRAM and DRAM.

The prediction database may be configured to receive and/or transmit data from/to any suitable device/module/database, including, but not limited to, modules residing in the controller. In some embodiments, the prediction database is configured to receive data from a detection module residing in the controller and/or from a detection database residing in the controller. In some embodiments, the prediction database is configured to transmit data to a prediction module residing in the controller.

In some embodiments, the controller comprises an instruction database configured to receive, store and/or transmit instructions for activating one or more electrodes (e.g, electrical signals captured by one or more electrodes inserted/implanted in the patient's brain).

The instruction database may comprise any suitable type of memory including, but not limited to, cache, ROM, PROM, EPROM, EEPROM, flash memory, SRAM and DRAM.

The instruction database may be configured to receive and/or transmit data from/to any suitable device/module/database, including, but not limited to, modules residing in the controller. In some embodiments, the instruction database is configured to receive data from a prediction module residing in the controller. In some embodiments, the instruction database is configured to transmit data to an activation module residing in the controller.

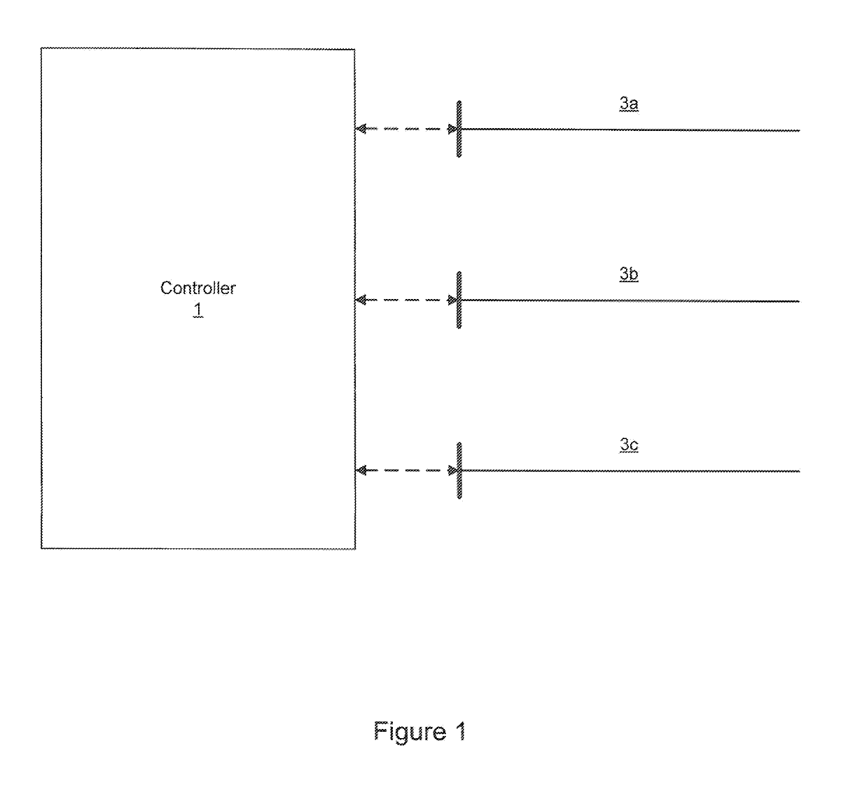

As shown in FIG. 1, in some embodiments, devices of the present invention comprise a controller 1 operatively connected to one or more electrodes 3a, 3b, 3c.

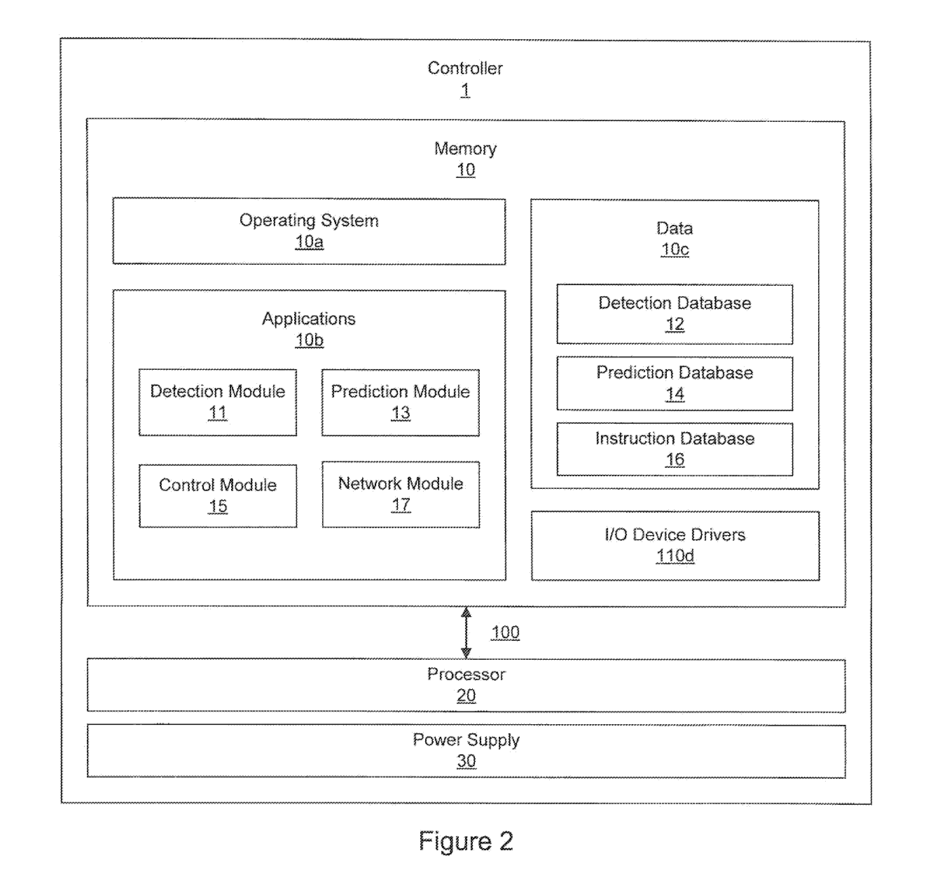

As shown in FIG. 2, in some embodiments, the controller 1 comprises memory 10, a processor 20 and a power supply 30 (e.g., an internal power supply), wherein memory 10 is representative of the overall hierarchy of memory devices containing software and data used to implement the functionality of the controller 1 and wherein the processor 20 communicates with the memory 10 via an address/data bus 100. In particular embodiments, memory 10 comprises an operating system 10a, applications 10b (e.g., a detection module 11 configured to receive data associated with the detection of neuronal activity in one or more regions of a patient's brain; a prediction module 13 configured to analyze data associated with the detection of neuronal activity in one or more regions of a patient's brain; an activation module 15 configured to activate one or more electrodes and/or a network module 17 configured to receive and/or transmit data), data 10c (e.g., a detection database 12 comprising data associated with data associated with the detection of neuronal activity in one or more regions of a patient's brain; a prediction database 14 comprising data associated with one or more predicted/detected cognitive activities and/or an instruction database 16 comprising instructions for activating one or more electrodes) and I/O drivers 100d.

### EXAMPLES

The following example is provided to illustrate some particular aspects of the present invention and is not intended to comprise a detailed catalog of all the different ways in which the present invention may be implemented or of all the features that may be incorporated into methods and apparatuses of the present invention.

### Example 1

### Stimulation of Hippocampal Neuronal Ensembles Restores Cognitive Function

Microelectrode recordings are collected from the hippocampus of patients already undergoing standard continuous monitoring for medically refractory epilepsy. Direct focal hippocampal stimulation is applied based on the recorded patterns for the recovery of cognitive function and facilitation of memory tasks.

### I. METHODS AND MEASURES

Potential participants undergo appropriate screening evaluations. Subjects included in the study 1) are 18 years to 75 years in age; 2) are able to give informed consent to participate in the study (consent is given by appropriate guardian if patient is unable); 3) have received a diagnosis of medically refractory epilepsy; 4) are candidates for and at willing to receive standard intracranial (i.e. Phase II) epilepsy monitoring for localization of seizure focus; 5) are able to participate in neurocognitive testing; 6) have completed evaluations that include cranial MRI and Phase I epilepsy monitoring; 7) are candidates for surgical treatment of epilepsy; 8) have no history of allergies to any materials to be used in the study; and 9) have verbal and nonverbal IQs in the low average range, or better, upon baseline assessment (completed in standard epilepsy protocol Phase I monitoring, or otherwise, within the past two years). Subjects are excluded from the study if they 1) are pregnant or lactating women (women of childbearing potential may be required to have a negative serum pregnancy test at screening); 2) have a clinically relevant abnormality on 12-lead electrocardiogram (ECG); 3) have evidence for other significant medical or psychiatric disorders, such as psychosis, frequent hallucinations, severe depression, significant cognitive decline in recent months, systemic organ failure, or bleeding diathesis which, in the opinion of the Investigator, could affect the subject's ability to tolerate or complete the study; 4) have significant concurrent or recently diagnosed (<2 months) medical condition that, in the opinion of the Investigator, could affect the subject's ability to tolerate or complete the study; 5) have serum platelets less than 100,000/mm3, International Normalized Ratio (INR) greater than 1.3 or partial thromboplastin time (PTT) greater than 40 seconds; or 6) have acute or chronic systemic and/or local skin infections involving the scalp.

Participants undergo implantation of FDA-approved hippocampal electrodes for diagnosis and localization of seizures using current practice for seizure localization. These patients additionally undergo neuronal ensemble recording and stimulation from microelectrodes contained within the implanted FDA-approved electrodes while performing approved neurocognitive tasks. Data recording during the post-implantation Phase II EMU monitoring and/or neurocognitive task performance is ceased for any evidence of neurologic deficit related to electrode placement, evidence of neurologic deficit or atypical seizure related to study-specific electrical stimulation, successful localization with planned perioperative cessation of recording, non-localization of seizure focus/foci after appropriate clinical time with appropriate clinical provocation as required, infection, refractory cerebrospinal fluid leakage, and/or imaging indicating sub-optimal placement of electrodes.

### II. NEUROCOGNITIVE TESTING

With hippocampal electrodes placed, measures of hippocampal-mediated learning and memory are collected according to standardized instructions. The Cambridge Neuropsychological Test Automated Battery (CANTAB) system is applied, using the Delayed Match to Sample (DMS) task which has shown clinically validity in the epilepsy population (Torgersen et al., 2012) as well as a custom-designed CANTAB-based visual DMS task (Deadwyler et al., 2007; Hampson et al., 2004; Hampson et al., 2011; Robert E Hampson and Greg, 2012) and a custom neuropsychological battery. During the acquisition (or, learning) phase of the trial, conformal, multineuron closed-loop-derived stimulation as well as control stimulation is applied in a controlled and selective fashion.

Using the CANTAB system, patients are presented test materials on a touch-sensitive screen and respond to items by touching the screen to demonstrate their ability at immediate sample to match, delayed sample to match and forced choice recognition. The software integrated into the system records and processes the responses to generate results as raw scores for the test. Raw scores from the CANTAB test can be compared to the mean raw scores in the reference population, allowing generation of z-scores and related cognitive performance. For the purpose of this study, it provide millisecond timing of the presentation of test items and patient response which is correlated to neuronal activity recorded from the invasive electrodes for analysis of neuronal ensembles.

Total time for stimulation administration is approximately 60-90 minutes per hippocampus; however, stimulation is episodic and is not continuously applied during that period. For each of the described tasks, an alternate form is used when available in order to minimize retest effects in future assessment.

### III. NEUROIMAGING

All patients routinely undergo post-implantation skull x-ray and MRI without contrast in order to confirm electrode placement. Depending on clinical status, patients may undergo repeat imaging during their Phase II evaluation. This could include MRI and/or CT imaging depending on clinical conditions and course. Additional imaging may also be performed on brain tissue resected as part of clinical seizure treatment.

### IV. LABORATORY ASSESSMENTS

Routine preoperative laboratory exams are analyzed by respective clinical chemistry and hematology laboratories. Postoperatively, the following parameters are assayed as needed per a standard Phase II clinical protocol: complete blood count (CBC) (including differential and platelet analysis); clotting parameters (prothrombin time, PTT, INR and fibrinogen level); and serum chemistry (sodium, potassium, chloride, total CO₂, blood urea nitrogen, glucose and creatinine).

### V. CONCOMITANT MEDICATION

Subjects continue to receive any medications that they were receiving at study entry for underlying medical conditions, with the exception that AEDs are decreased or discontinued per standard protocol to allow seizure localization. Subjects are on stable medication (2 months) for underlying medical conditions. Changes in AEDs are documented appropriately and post-operative pain medications are given as needed.

Patients on anticoagulation therapy are able to maintain, with acceptable risk, a normal INR and PTT for at least 1 week before surgery and 3 weeks after surgery as determined by the patient's primary care physician. Any patient receiving aspirin is able to discontinue this drug at least 1 week prior to surgery and for 3 weeks following surgery.

Subjects are prohibited from receiving other investigational therapies or undergoing other brain surgery during participation in this study.

### VII. OUTCOME MEASURES

The primary endpoint of this trial is completion of at least a single session of neuronal ensemble recordings and stimulation while performing described neurocognitive tasks, and at least a single session of baseline recording. When clinically appropriate, single-unit recording likely is continuous throughout the stay within the EMU to better develop models of baseline hippocampal function. These sessions is performed either in sequence with or parallel to standard, invasive, Phase II monitoring for localization of epileptic foci. Importantly, duration of electrode placement is not increased by adding these additional recordings. Electrodes are removed when seizure foci have been adequately localized per our current clinical protocol.

A general Volterra kernel-based strategy for modeling nonlinear dynamics underlying spike train-to-spike train transformations between CA3 and CA1 is established to predict output patterns of CA1 firing pattern from input patterns of CA3 neural activity (Song et al., 2009). In this approach, the identification of spatio-temporal pattern transformations from the hippocampal CA3 region to the CA1 region is formulated as the estimation of a conformal multineuron closed-loop model that can be decomposed into a series of component models with physiologically identifiable structure that can be expressed by the following equations: w=u(k, x)+a(h, y)+ε(σ), y=0 when w<θ and 1 when w>θ. The variable x represents input spike trains; y represents output spike trains. The hidden variable w represents the pre-threshold membrane potential of the output neurons, and is equal to the summation of three components, i.e. postsynaptic potential u caused by input spike trains, the output spike-triggered after-potential a, and a Gaussian white noise s with standard deviation a. The noise term models both intrinsic noise of the output neuron and the contribution of unobserved inputs. When w exceeds threshold, θ, an output spike is generated and a feedback after-potential (a) is triggered and then added to w. Feedforward kernels k describe the transformation from x to u. The feedback kernel, h, describes the transformation from y to a as previously described (Berger et al., 2011). All model parameters, (i.e., feedforward Volterra kernel k and feedback Volterra kernel h) can be estimated using an iterative re-weighted least-squares method (Truccolo et al., 2005). Noise standard deviation σ and threshold θ are redundant variables and thus can be indirectly obtained through variable transformation (Song et al., 2009). Due to the stochastic nature of the system, estimated models are validated using an out-of-sample Kolmogorov-Smirnov test based on the time-rescaling theorem (Brown et al., 2002). As stated above, we anticipate recording data from approximately 10 patients to establish and validate the model in humans.

Demographic and baseline characteristics are summarized using standard methods and statistics (mean, median, standard deviation, frequency, and percentage).

### VI. SCHEDULE

The study schedule is summarized in Table I below.

TABLE 1
Schedule of Procedures and Evaluations by Visit

| | Visit | | | | | | | | |
|---|---|---|---|---|---|---|---|---|---|
| | 1 | 2 | 3 | | 4 | 5 | 6 | 7 | 8 |
| | Day (d)/Month (m) | | | | | | | | |
| | | | Surgery/Hospital | | | | | | |
| | −270 d | −35 d | | Up to | | | | | |
| | to −90 d | to −21 d | 0 d | 14 d | 14 d ± 7 d | 28 d ± 7 d | 3 m ± 5 d | 6 m ± 5 d | 12 m ± 5 d |
| Informed consent | | X | | | | | | | |
| Inclusion/exclusion criteria | | X | | | | | | | |
| Demographics | | X | | | | | | | |
| Medical/surgical history | | X | X | | | | | | |
| Previous and concomitant | | X | X | | X | X | X | X | X |
| medications^{A} | | | | | | | | | |
| Weaning of antiepileptic | | | | X | | | | | |
| medications for seizure | | | | | | | | | |
| localization | | | | | | | | | |
| Physical examination^{B} | | X | X | X | X | X | X | X | X |
| Vital signs and complete | | X | X^{C} | X | X | X | X | X | X |
| neurological examination | | | | | | | | | |
| ECG | | X | | | X | | | | |
| Laboratory assessments^{D} | | X | | X | X | X | X | X | X |
| Serum pregnancy test | | X | | | | | | | |
| MRI of brain | X^{E} | | X | | | | | X | X |
| Skull Xray for confirmation of | | | X | | | | | | |
| electrode placement | | | | | | | | | |
| CT or MRI for pre-surgical | X^{E} | | | | | | | | |
| planning | | | | | | | | | |
| MEG for pre-surgical planning | X | | | | | | | | |
| SPECT for pre-surgical planning | X | | | | | | | | |
| (if practical) | | | | | | | | | |
| PET for press-surgical planning | X | | | | | | | | |
| (if practical) | | | | | | | | | |
| Surgery and related procedures | | | X | X | | | | | |
| Seizure Localization | | | | X | | | | | |
| Hippocampal Neuronal Ensemble | | | | X^{F} | | | | | |
| Recordings and Stimulation | | | | | | | | | |
| Removal of electrodes and epilepsy | | | | X | | | | | |
| surgery as indicated | | | | | | | | | |
| Subject follow-up in hospital (Up | | | X | X | | | | | |
| to Day 14) | | | | | | | | | |
| Remove sutures and assess surgical | | | | | X | | | | |
| sites | | | | | | | | | |
| Clinical follow-up | | | | | | X | X | X | X |

^{A}Previous and concomitant medications are recorded at Visit 1 and any changes recorded at subsequent visits.
^{B}A complete physical examination is obtained at Visit 1 and symptom-directed physical examinations are recorded at subsequent visits.
^{C}Following completion of electrode implantation and again after resective surgery, the subject is transferred to in-patient hospital service where vital signs are recorded every 1 hour for the first night following surgery, and then every 4-8 hours for the next 48 hour.
^{D}CBC, including differential and platelet analysis; clotting parameters (prothrombin time, PTT, INR and fibrinogen level); and serum chemistry (sodium, potassium, chloride, total CO₂, blood urea nitrogen, glucose and creatinine).
^{E}MRI and CT scans done for surgical screening are used as baseline assessments.
^{F}Neurocognitive testing, stimulation.

### VIII. REFERENCES

Aggleton, J. P., and Brown, M. W. (1999). Episodic memory, amnesia, and the hippocampal-anterior thalamic axis. Behavioral and Brain Sciences 22, 425-444.
Agnew, W. F., and McCreery, D. B. (1990). Considerations for Safety with Chronically Implanted Nerve Electrodes. Epilepsia 31, S27-S32.
Berger, T. W., Hampson, R. E., Song, D., Goonawardena, A., Marmarelis, V. Z., and Deadwyler, S. A. (2011). A cortical neural prosthesis for restoring and enhancing memory. Journal of Neural Engineering 8, 046017.
Bemier, G. P., Richer, F., Giard, N., Bouvier, G., Mercier, M., Turmel, A., and Saint-Hilaire, J. M. (1990). Electrical stimulation of the human brain in epilepsy. Epilepsia 31, 513-520.
Boon, P., Vonck, K., De, H., V, Van, D. A., Goethals, M., Goossens, L., Van, Z. M., De, S. T., Dewaele, I., Achten, R., Wadman, W., Dewaele, F., Caemaert, J., and Van, R. D. (2007). Deep brain stimulation in patients with refractory temporal lobe epilepsy. Epilepsia 48, 1551-1560.
Brown, E. N., Barbieri, R., Ventura, V.r., Kass, R. E., and Frank, L. M. (2002). The Time-Rescaling Theorem and Its Application to Neural Spike Train Data Analysis. Neural Computation 14, 325-346.
Deadwyler, S. A., Porrino, L., Siegel, J. M., and Hampson, R. E. (2007). Systemic and Nasal Delivery of Orexin-A (Hypocretin-1) Reduces the Effects of Sleep Deprivation on Cognitive Performance in Nonhuman Primates. Journ. of Neurosci. 27, 14239-14247.
Duvernoy, H. M. (2005). The Human Hippocampus: Functional Anatomy, Vascularization and Serial Sections with MRI (Berlin Heidelberg, Germany: Springer).
Eichenbaum, H. (2004). Hippocampus: cognitive processes and neural representations that underlie declarative memory. Neuron 44, 109-120.
Engel, J., Jr., Wiebe, S., French, J., Sperling, M., Williamson, P., Spencer, D., Gumnit, R., Zahn, C., Westbrook, E., and Enos, B. (2003a). Practice parameter: temporal lobe and localized neocortical resections for epilepsy. Epilepsia 44, 741-751.
Engel, J., Jr., Wiebe, S., French, J., Sperling, M., Williamson, P., Spencer, D., Oumnit, R., Zahn, C., Westbrook, E., and Enos, B. (2003b). Practice parameter: temporal lobe and localized neocortical resections for epilepsy: report of the Quality Standards Subcommittee of the American Academy of Neurology, in association with the American Epilepsy Society and the American Association of Neurological Surgeons. Neurology 60, 538-547.
Feindel, W. (1998). Brain stimulation combined with electrocorticography in the surgery of epilepsy: historical highlights. Electroencephalogr. Clin. Neurophysiol. Suppl 48, 1-8.
Fried, I., Katz, A., McCarthy, G., Sass, K. J., Williamson, P., Spencer, S. S., and Spencer, D. D. (1991). Functional organization of human supplementary motor cortex studied by electrical stimulation. J. Neurosci. 11, 3656-3666.
Gordon, B., Lesser, R. P., Rance, N. E., Hart, J., Webber, R., Uematsu, S., and Fisher, R. S. (1990). Parameters for direct cortical electrical stimulation in the human: histopathologic confirmation. Electro. Clin. Neurophysiol. 75, 371-377.
Guzowski, J. F., Knierim, J. J., and Moser, E. I. (2004). Ensemble Dynamics of Hippocampal Regions CA3 and CA1. Neuron 44, 581-584.
Hamani, C., McAndrews, M. P., Cohn, M., Oh, M., Zumsteg, D., Shapiro, C. M., Wennberg, R. A., and Lozano, A. M. (2008). Memory enhancement induced by hypothalamic/formix deep brain stimulation. Ann. Neurol. 63, 119-123.
Hampson, R. E., Dong, S., Chan, R. H. M., Sweatt, A. J., Riley, M. R., Gerhardt, G. A., Shin, D. C., Marmarelis, V. Z., Berger, T. W., and Deadwyler, S. A. (2012). A Nonlinear Model for Hippocampal Cognitive Prosthesis: Memory Facilitation by Hippocampal Ensemble Stimulation. Neural Systems and Rehabilitation Engineering, IEEE Transactions on 20, 184-197.
Hampson, R. E., Pons, T. P., Stanford, T. R., and Deadwyler, S. A. (2004). Categorization in the monkey hippocampus: A possible mechanism for encoding information into memory. Proceedings of the National Academy of Sciences of the United States of America 101, 3184-3189.
Hampson, R., Porrino, L., Opris, I., Stanford, T., and Deadwyler, S. (2011). Effects of cocaine rewards on neural representations of cognitive demand in nonhuman primates. Psychopharmacology 213, 105-118.
Iadarola, M. J., and Gale, K. (1982). Substantia nigra: site of anticonvulsant activity mediated by gamma-aminobutyric acid. Science 218, 1237-1240.
Jensen, A. L., and Durand, D. M. (2007). Suppression of axonal conduction by sinusoidal stimulation in rat hippocampus in vitro. J Neural Eng 4, 1-16.
Jobst, B. C. (2010). Electrical stimulation in epilepsy: vagus nerve and brain stimulation. Curr. Treat. Options. Neurol. 12, 443-453.
Kesner, R. P., and Hopkins, R. O. (2001). Short-term memory for duration and distance in humans: Role of the hippocampus. Neuropsychology 15, 58-68.
Kwan, P., and Brodie, M. J. (2000). Early identification of refractory epilepsy. N. Engl. J. Med. 342, 314-319.
Laxton, A. W., Tang-Wai, D. F., McAndrews, M. P., Zumsteg, D., Wennberg, R., Keren, R., Wherrett, J., Naglie, G., Hamani, C., Smith, G. S., and Lozano, A. M. (2010). A phase I trial of deep brain stimulation of memory circuits in Alzheimer's disease. Ann. Neurol. 68, 521-534.
Lee, S. K., Lee, S. Y., Kim, K. K., Hong, K. S., Lee, D. S., and Chung, C. K. (2005). Surgical outcome and prognostic factors of cryptogenic neocortical epilepsy. Ann. Neurol. 58, 525-532.
Lian, J., Bikson, M., Sciortino, C., Stacey, W. C., and Durand, D. M. (2003). Local suppression of epileptiform activity by electrical stimulation in rat hippocampus in vitro. J. Physiol 547, 427-434.
Loddenkemper, T., Pan, A., Neme, S., Baker, K. B., Rezai, A. R., Dinner, D. S., Montgomery, E. B., Jr., and Luders, H. O. (2001). Deep brain stimulation in epilepsy. J. Clin. Neurophysiol. 18, 514-532.
Mark Witcher, and Thomal L. Ellis (2011). Neuromodulatory Treatment of Medically Refractory Epilepsy. In Novel Treatment of Epilepsy, Humberto Foyaca-Sibat, ed. (Rijeka, Croatia: InTech), pp. 287-310.
Matsumoto, R., Nair, D. R., LaPresto, E., Najm, I., Bingaman, W., Shibasaki, H., and Luders, H. O. (2004). Functional connectivity in the human language system: a cortico-cortical evoked potential study. Brain 127, 2316-2330.
MELTZER, T., and REDER, E. L. (1950). Metrazol and electric stimulation as diagnostic aids in epilepsy. N.Y. State J. Med. 50, 319-322.
Morrell, M. J. (2011). Responsive cortical stimulation for the treatment of medically intractable partial epilepsy. Neurology 77, 1295-1304.
Mueller, W. M., and Morris, G. L., III (1993). Intraoperative and extraoperative identification of eloquent brain using stimulation mapping. Neurosurg. Clin. N. Am. 4, 217-222.
Robert E Hampson and Greg (2012). Facilitation and restoration of cognitive function in primate prefrontal cortex by a neuroprosthesis that utilizes minicolumn-specific neural firing. Journal of Neural Engineering 9, 056012.
Sander, J. W. (1993). Some aspects of prognosis in the epilepsies: a review. Epilepsia 34, 1007-1016.
Schaffer, K. (1892). Beitrag zur histologie der ammonshornformation). Arch mikroskop Anatomic 39, 611-632.
Scoville, W. B., and Milner, B. (1957). Loss of recent memory after bilateral hippocampal lesions. J. Neurol. Neurosurg. Psychiat. 20, 11-21.
Sillanpaa, M., and Schmidt, D. (2006). Prognosis of seizure recurrence after stopping antiepileptic drugs in seizure-free patients: A long-term population-based study of childhood-onset epilepsy. Epilepsy Behav. 8, 713-719.
Smith, D. M., and Mizumori, S. J. Y. (2006). Hippocampal place cells, context, and episodic memory. Hippocampus 16, 716-729.
Song, D., Chan, R. H. M., Marmarelis, V. Z., Hampson, R. E., Deadwyler, S. A., and Berger, T. W. (2009). Nonlinear modeling of neural population dynamics for hippocampal prostheses. Neural Networks 22, 1340-1351.
Squire, L. R., Stark, C. E., and Clark, R. E. (2004). The medial temporal lobe. Annu. Rev. Neurosci 27, 279-306.
Squire, L. R., and Zola-Morgan, S. (1991). The medial temporal lobe memory system. Science 253, 1380-1386.
Sramka, M., Fritz, G., Gajdosova, D., and Nadvornik, P. (1980). Central stimulation treatment of epilepsy. Acta Neurochir. Suppl (Wien.) 30, 183-187.
Suthana, N., Haneef, Z., Stern, J., Mukamel, R., Behnke, E., Knowlton, B., and Fried, I. (2012). Memory Enhancement and Deep-Brain Stimulation of the Entorhinal Area. N Engl J Med 366, 502-510.
Tellez-Zenteno, J. F., McLachlan, R. S., Parrent, A., Kubu, C. S., and Wiebe, S. (2006). Hippocampal electrical stimulation in mesial temporal lobe epilepsy. Neurology 66, 1490-1494.
Theodore, W. H. (2005). Brain stimulation for epilepsy. Nat. Clin. Pract. Neurol. 1, 64-65.
Theodore, W. H., and Fisher, R. (2007). Brain stimulation for epilepsy. Acta Neurochir. Suppl 97, 261-272.
Torgersen, J., Flaatten, H., Engelsen, B., and Gramstad, A. (2012). Clinical Validation of Cambridge Neuropsychological Test Automated Battery in a Norwegian Epilepsy Population. Journal of Behavioral and Brain Science 2, 108-116.
Truccolo, W., Eden, U. T., Fellows, M. R., Donoghue, J. P., and Brown, E. N. (2005). A Point Process Framework for Relating Neural Spiking Activity to Spiking History, Neural Ensemble, and Extrinsic Covariate Effects. Journal of Neurophysiology 93, 1074-1089.
Van, R. D., Boon, P., Vonck, K., and Caemaert, J. (2007). Neurosurgical aspects of temporal deep brain stimulation for epilepsy. Acta Neurochir. Suppl 97, 333-336.
Velasco, F., Velasco, M., Jimenez, F., Velasco, A. L., and Marquez, I. (2001a). Stimulation of the central median thalamic nucleus for epilepsy. Stereotact. Funct. Neurosurg. 77, 228-232.
Velasco, F., Velasco, M., Velasco, A. L., Jimenez, F., Marquez, I., and Rise, M. (1995). Electrical stimulation of the centromedian thalamic nucleus in control of seizures: long-term studies. Epilepsia 36, 63-71.
Velasco, F., Velasco, M., Velasco, A. L., Menez, D., and Rocha, L. (2001b). Electrical stimulation for epilepsy: stimulation of hippocampal foci. Stereotact. Funct. Neurosurg. 77, 223-227.
Wellmer, J.+., von der Groeben, F., Klarmann, U., Weber, C., Elger, C. E., Urbach, L., Clusmann, H., and von Lehe, M. (2012). Risks and benefits of invasive epilepsy surgery workup with implanted subdural and depth electrodes. Epilepsia 53, 1322-1332.
Zanos, T. P., Courellis, S. H., Berger, T. W., Hampson, R. E., Deadwyler, S. A., and Marmarelis, V. Z. (2008). Nonlinear Modeling of Causal Interrelationships in Neuronal Ensembles. Neural Systems and Rehabilitation Engineering, IEEE Transactions on 16, 336-352.

The foregoing is illustrative of the present invention, and is not to be construed as limiting thereof. The invention is defined by the following claims, with equivalents of the claims to be included herein.

1. A method, comprising:
detecting neuronal activity in the CA3 region of a patient's hippocampus; and
analyzing the detected neuronal activity to predict and/or detect a cognitive activity.

2. A method, comprising:
detecting neuronal activity in the CA3 region of a patient's hippocampus and at least one alternate region of the patient's brain; and
analyzing the detected neuronal activity to predict and/or detect a cognitive activity.

3. The method of claim 2, wherein said at least one alternate region of the patient's brain comprises the Schaffer collateral region of the hippocampus, the CA1 region of the hippocampus, the CA2 region of the hippocampus, the perforant pathway, the subiculum, the entorhinal cortex and/one or more neocortical regions.

4. The method of claim 1, wherein said detecting step comprises using one or more electrodes to detect electrical impulses generated by one or more neurons.

5. The method claim 1, wherein said detecting step comprises detecting electrical impulses using one or more electrodes configured to detect electrical impulses generated by a single neuron.

6. The method claim 1, wherein said detecting step comprises detecting electrical impulses using one or more electrodes configured to detect electrical impulses generated by a plurality of neurons.

7. The method claim 1, wherein said analyzing step comprises predicting future neuronal activity based upon the detected neuronal activity.

8. The method of claim 7, wherein predicting future neuronal activity based upon the detected neuronal activity comprises the use of a predictive algorithm.

9. The method of claim 1, wherein said analyzing step comprises comparing the detected neuronal activity with neuronal activity that was detected in the CA3 region of the patient's hippocampus and/or the at least one alternate region prior to and/or during a previous cognitive activity.

10. The method of claim 1, wherein said analyzing step comprises comparing the detected neuronal activity with neuronal activity that was detected in the CA3 region and/or the at least one alternate region of one or more others patients prior to and/or during a cognitive activity.

11. The method of claim 1, wherein said analyzing step comprises comparing the detected neuronal activity with a neuronal activity signature associated with one or more types of cognitive activity.

12. The method of claim 1, wherein said analyzing step comprises analyzing the detected neuronal activity using a computer program product comprising computer-readable code embodied in a computer-readable non-transient storage medium.

13. A method, comprising:
predicting and/or detecting a cognitive activity in a patient; and
delivering a stimulus to one or more regions of the patient's brain to improve cognitive function.

14. The method of claim 13, wherein said predicting and/or detecting step comprises the method of claim 1.

15. The method of claim 13, wherein a stimulus is delivered to the CA3 region of the hippocampus, the Schaffer collateral region of the hippocampus, the CA1 region of the hippocampus, the CA2 region of the hippocampus, the perforant pathway, the subiculum, the entorhinal cortex, the cerebellum and/or one or more neocortical regions.

16. The method of claim 13, wherein delivering a stimulus to one or more regions of the patient's brain to improve cognitive function comprises delivering one or more electrical impulses to each of the stimulated regions.

17. The method of claim 13, wherein said predicting and/or detecting step comprises analyzing neuronal activity using a computer program product comprising computer-readable code embodied in a computer-readable non-transient storage medium.

18. The method of claim 13, wherein said delivering step comprises using one or more electrodes to deliver one or more electrical impulses to the patient's brain.

19. A device comprising:
a controller operatively connected to one or more electrodes,
wherein at least one of said one or more electrodes is configured to detect neuronal activity in the CA3 region of a patient's hippocampus, and
wherein said controller is configured improve cognitive function based upon the neuronal activity detected in the CA3 region of a patient's hippocampus.

20. The device of claim 19, wherein at least one of said one or more electrodes is configured to deliver an electrical stimulus to the CA1 region of the patient's hippocampus, and
wherein said controller is further configured to activate one or more of the electrodes configured to deliver an electrical stimulus to the CA1 region of the patient's hippocampus responsive to predicting and/or detecting the cognitive activity.

21. A device comprising:
a controller operatively connected to a plurality of electrodes,
wherein one or more of said plurality of electrodes is configured to detect neuronal activity in the CA3 region of a patient's hippocampus,
wherein one or more of said plurality of electrodes is configured to detect neuronal activity in an alternate region of the patient's brain, and
wherein said controller is configured to detect a cognitive activity based upon the neuronal activity detected in the CA3 region of a patient's hippocampus and the alternate region of the patient's brain.

22. The device of claim 21, wherein one or more of said plurality of electrodes is configured to deliver an electrical stimulus to the CA1 region of the patient's hippocampus, and
wherein said controller is further configured to activate one or more of the electrodes configured to deliver an electrical stimulus to the CA1 region of the patient's hippocampus responsive to predicting and/or detecting the cognitive activity.

23. The device of claim 21, wherein one or more of said plurality of electrodes is configured to deliver an electrical stimulus to the alternate region of the patient's brain, and
wherein said controller is further configured to activate one or more of the electrodes configured to deliver an electrical stimulus to the alternate region of the patient's brain responsive to predicting and/or detecting the cognitive activity.

24. The device of claim 21, wherein said alternate region of the patient's brain comprises the Schaffer collateral region of the patient's hippocampus, the CA3 region of the hippocampus, the CA2 region of the hippocampus, the perforant pathway, the subiculum, the entorhinal cortex, the cerebellum and/or one or more neocortical regions.

25. The device of claim 19, wherein said controller is configured to predict future neuronal activity based upon the detected neuronal activity.

26. The device of claim 25, wherein said controller is configured to predict future neuronal activity based upon the detected neuronal activity using of a predictive algorithm.

27. The device of claim 19, wherein said controller is configured to compare the detected neuronal activity with neuronal activity that was detected in the CA3 region of the patient's hippocampus and/or the at least one alternate region prior to and/or during a previous cognitive activity.

28. The device of claim 1, wherein said controller is configured to compare the detected neuronal activity with neuronal activity that was detected in the CA3 region and/or the at least one alternate region of one or more others patients prior to and/or during a cognitive activity.

29. The device of claim 19, wherein said controller is configured to compare the detected neuronal activity with a neuronal activity signature associated with one or more types of cognitive activity.

30. The device of claim 20, wherein activation of said one or more of the electrodes configured to deliver an electrical stimulus to the CA1 region of the patient's hippocampus enhances the propagation of neuronal activity in the patient's hippocampus.

31. The device of claim 23, wherein activation of said one or more of the electrodes configured to deliver an electrical stimulus to the alternate region of the patient's brain enhances the propagation of neuronal activity in the patient's hippocampus.

32-33. (canceled)
